(19) United States
(12) Patent Application Publication
(10) Pub. No.: US 2023/0181491 A1
(43) Pub. Date: Jun. 15, 2023

(54) COMPOSITIONS AND METHODS FOR THE TREATMENT AND MANAGEMENT OF INFLAMMATION USING HYDROXYNORKETAMINE

(71) Applicant: SPIRIFY PHARMA INC., Saddle Brook, NJ (US)

(72) Inventors: Sigal KREMER-TAL, Ramat Hasharon (IL); Irving William WAINER, Washington, DC (US); Joseph PERGOLIZZI, Naples, FL (US)

(21) Appl. No.: 17/925,435

(22) PCT Filed: May 19, 2021
(86) PCT No.: PCT/IL2021/050574

§ 371 Date: Nov. 15, 2022

Related U.S. Application Data
(60) Provisional application No. 63/027,364, filed on May 20, 2020.

(51) Int. Cl. A61K 31/135; A61P 31/14

(52) U.S. Cl. CPC A61K 31/135; A61P 31/14

(57) ABSTRACT

The present invention pertains to compositions and methods for treating inflammation in a subject in need thereof using hydroxynorketamine (HNK), a salt thereof, a stereoisomer thereof, or a combination thereof.

### CROSS REFERENCE TO RELATED APPLICATION

This application claims priority from U.S. Provisional Application No. 63/027,364, filed May 20, 2020, which is hereby incorporated by reference in its entirety.

### FIELD OF THE INVENTION

The present invention pertains to methods and compositions for the treatment of inflammation using hydroxynorketamine.

### BACKGROUND OF THE INVENTION

(R,S)-Ketamine is a phenylcyclohexylamine derivative consisting of two optical enantiomers, (S)- and (R)-ketamine (hereinafter collectively referred to as ketamine). Ketamine is an effective anesthetic agent that does not produce significant cardiovascular or pulmonary effects. Sub-anesthetic doses of the drug are widely used as non-opioid analgesic for peripheral pain, neuropathic pain associated with complex regional pain syndrome (CRPS) and migraine headache. It is also prescribed to reduce perioperative opioid usage and in the clinical treatment of depression, suicidal ideation, and post-traumatic stress syndrome (PTSD). The racemic, 50:50 ketamine mixture, i.e., (R,S)-ketamine, is the predominately used formulation, although the FDA has recently approved an intranasal formulation containing (S)-ketamine for the treatment of depression. (S)-Ketamine is also widely used for pain indications.

Ketamine is rapidly and extensively metabolized into a wide range of N-demethylated, hydroxylated and unsaturated compounds and their respective enantiomeric and diastereomeric isomers. Ketamine undergoes extensive metabolism, initially via nitrogen demethylation to norketamine (NK), a reaction that is catalyzed primarily by the cytochrome P450 liver enzymes CYP2B6 and CYP3A4. Following demethylation of ketamine to norketamine, norketamine is further metabolized to the hydroxynorketamines (HNKs) and dehydronorketamine (DHNK). Metabolism to (2R,6R;2S,6S)-HNK is primarily carried out by CYP2A6 and CYP2B6. These enzymes are also responsible for the formation of the (2S,4S;2R,4R)- and (2S,5S;2R,5R)-HNKs. CYP3A4 and CYP3A5 are the principal enzymes identified to catalyze the conversion of norketamine to (2S,4R;2R,4S)-HNK, whereas CYP2B6 is predominantly responsible for the catalysis of the conversion of norketamine to (2S,5R;2R,5S)-HNK. The other secondary metabolite is DHNK. DHNK is directly formed from norketamine primarily via the actions of the CYP2B6 enzyme, or from 5-HNK via a nonenzymatic dehydration event. In addition to the major metabolic pathways of ketamine, there are several other pathways that have also been studied such as hydroxylation of ketamine to 6-hydroxyketamine (HK). Further, phenolic isomers of HNKs have also been observed, potentially resulting from the hydroxylation of norketamine (Panos Zanos et al., Pharmacol. Rev. 2018 Jul;70(3):621-660).

The activity of ketamine metabolites is yet to be elucidated and, in some cases, controversial. Pharmacodynamic studies of ketamine conducted in rats examined the anesthetic effects of this agents and its two principal metabolites, (R,S)-norketamine and (2R,6R;2S,6S)-HNK. The results demonstrated that (R,S)-ketamine and (R,S)-norketamine produced anesthetic actions and increased spontaneous locomotor activity during the postanesthetic recovery phase, whereas (2R,6R;2S,6S)-HNK had no anesthetic or hyperlocomotor effects. Other studies demonstrated that the (2S,6S)- HNK and (2R,6R)-HNK metabolites exert antidepressant-relevant behavioral responses in rodents (Panos Zanos et al., Pharmacol. Rev. 2018 Jul;70(3):621-660). Panos Zanos et al. (Nature volume 533, pages 481-486; 2016) illustrated that while ketamine is a NMDAR antagonist, its metabolite (2R,6R)-HNK exerts behavioral, electroencephalographic, electrophysiological and cellular antidepressant-related actions in mice via activation of AMPARs (α-amino-3-hydroxy-5-methyl-4-isoxazole propionic acid receptors).

Inflammation is a critical homeostatic mechanism used by the body to fight infections and to heal tissue injuries. Inflammatory reactions are triggered once immune cells of the innate immune system become activated, whether by invading pathogens or tissue damage. Release of proinflammatory cytokines by these cells then activates members of the adaptive immune system to initiate an inflammatory response.

Recent clinical, animal and cell-based data suggests that ketamine has anti-neuroinflammatory activity associated with a reduction in the production of cytokines and chemokines (Yanning Li et al., Frontiers in Pharmacology, Volume 8, Article 139; 20 Mar. 2017). Additional data suggests that S-ketamine attenuates surgery-associated increase in TNF and IL-6 in radical prostatectomy patients (G. Shaked et al., Resuscitation 62 (2004) 237-242). Anti-inflammatory actions (i.e., reduction of proinflammatory cytokines) of preoperative subanesthetic doses of 0.15-0.25 mg/kg (single i.v. bolus) ketamine were described in humans. Ketamine was shown to inhibit immune reaction-induced proinflammatory cytokine production, including nuclear factor kB, and to decrease blood levels of tumor necrosis factor-a, interleukin 6 (IL-6), C-reactive protein, and/or inducible nitric oxide synthase (Panos Zanos et al., Pharmacol. Rev. 2018 Jul;70(3):621-660).

The clinical use of ketamine is limited due to the preferred route of administration (i.e., i.v.) and its side effects. The side effects of ketamine inter alia include anxiety, agitation or irritability, euphoria or mood elevation, delusions or unusual thoughts, panic, apathy, dissociation, perceptual disturbance, odd or abnormal sensation, derealization, hallucinations, feeling strange, weird, bizarre, or unreal, and depersonalization. Additional side effects include increased blood pressure, increased heart rate, headaches and dizziness (K. Hirota, and D. G. Lambert; British Journal of Anesthesia, 121 (6): 1198e1202 (2018).

Recent studies demonstrated that hydroxynorketamine (HNK) is a rapidly produced and major metabolite of ketamine, which produces many of the therapeutic effects attributed to ketamine in the treatment of depression and neuropathic pain. Both (2R,6R)-HNK and (2S,6S)-HNK are pharmacologically active and do not produce the same dissociative effects attributed to ketamine. HNK isomers are orally bioavailable and exhibit low toxicity and favorable distribution and elimination profiles. The effect on intracellular and mitochondrial signaling has been determined and the data indicate that treatment with (2R,6R)-HNK and (2S,6S)-HNK produces increased signaling via the mTOR pathway.

U.S. Pat. No. 9,650,352 to an inventor of the present invention discloses pharmaceutical preparations containing (2R,6R)-hydroxynorketamine, or (R)- or (5)-dehydronorketamine, or other stereoisomeric dehydro or hydroxylated ketamine metabolite. The disclosure provides methods of treating bipolar depression, major depressive disorder, neuropathic and chronic pain, including complex regional pain disorder (CRPS) by administering a purified ketamine metabolite or a ketamine metabolite prodrug directly to patients in need of such treatment, the content of which is incorporated herein by reference.

International patent application, publication No. WO/2017/087388 to an inventor of the present invention discloses phenyl cyclohexanone based active agents, pharmaceutical preparations containing such active agents, methods of modifying cellular activity by contacting cells with such active agents, and methods of treating various conditions by administering such active agents to a patient, the content of which is incorporated herein by reference.

Coronaviruses are a group of related RNA viruses that cause diseases (e.g., Coronavirus disease 2019; COVID-19) in mammals and birds. In humans, these viruses cause respiratory tract infections that can range from mild to lethal. They are enveloped viruses with a positive-sense single-stranded RNA genome and a nucleocapsid of helical symmetry.

Secondary haemophagocytic lymphohistiocytosis (sHLH) is an under-recognized, hyperinflammatory syndrome characterized by a fulminant and fatal hyper-cytokinemia, causing multiorgan failure. In adults, sHLH is most commonly triggered by viral infections. Cardinal features of sHLH include unremitting fever, cytopenias, and hyper-ferritinaemia. Further, pulmonary involvement, including acute respiratory distress syndrome (ARDS), occurs in approximately 50% of patients afflicted with sHLH.

Accumulating data demonstrate a cytokine profile associated with COVID-19 disease severity which resembles the profile of sHLH. This cytokine profile is characterized by increased interleukin IL-2, IL-7, granulocyte colony stimulating factor (G-CSF), interferon-γ inducible protein 10 (CXCL10), monocyte chemoattractant protein 1 (MCP-1), macrophage inflammatory protein 1-α (MIP-1α), and tumor necrosis factor-α (TNF-α). Predictors of fatality from a recent retrospective, multicenter study of 150 confirmed COVID-19 cases in Wuhan, China, included elevated ferritin and IL-6 (p<0·0001), suggesting that mortality might be due to virally driven hyperinflammation (Mehta P. et al., the Lancet, Vol. 395, p. 1033-1034).

There is an immediate need in anti-inflammatory drugs that would be effective yet safe and would allow the management and treatment of inflammation. The present invention suggests the use of HNK for attenuating inflammation.

### SUMMARY OF THE INVENTION

The present invention pertains to methods for the treatment and management of inflammation in a subject comprising administering to a subject in need of such treatment a pharmaceutical composition comprising hydroxynorketamine (HNK), a stereoisomer thereof or a salt thereof. The present invention further pertains to methods of inhibiting proinflammatory agents (e.g., cytokines) secretion from immune cells (e.g., monocytes) comprising contacting cells of the immune system with HNK, thereby inhibiting the proinflammatory agents secretion from the immune cells.

The present invention is based in part on the unexpected discovery that HNK, in contrast to other ketamine metabolites, effectively inhibits proinflammatory agents' secretion (e.g., IL-6 and TNF-α) from monocytes and exhibits safety to the monocytes. Specifically, the herein inventors revealed that while a certain ketamine metabolite, i.e., dehydronorketamine (DHNK) is toxic to monocyte cells, HNK is safe to those cells and does not cause any monocyte cells' mortality. Further, the ketamine metabolite R,S-norketamine (NK) acts to promote inflammation by inducing the secretion of the proinflammatory agent Prostaglandin E2 (PGE2) from monocyte cells. As to ketamine, this agent acts as a double-edged sword in the treatment and management of inflammation. Ketamine, while possessing anti-inflammatory properties, is metabolized in the body to form NK (which herein demonstrated to induce PGE2 secretion) and DHNK (which herein demonstrated to be toxic to monocytes). Further, ketamine administration to patients is known to be associated with some serious side effects. Accordingly, the herein invention suggests the use of HNK which is both safe and effective in the treatment of inflammation.

According to a first aspect the present invention provides a method of treating or attenuating inflammation in a subject in need thereof; the method comprising administering to the subject a pharmaceutical composition comprising a therapeutically effective amount of hydroxynorketamine (HNK), a pharmaceutically acceptable salt thereof, a stereoisomer thereof, or a combination thereof, and a pharmaceutical acceptable carrier, thereby treating inflammation in the subject.

According to a second aspect, the present invention provides a pharmaceutical composition comprising a therapeutically effective amount of HNK, a pharmaceutically acceptable salt thereof, a stereoisomer thereof, or a combination thereof, and a pharmaceutical acceptable carrier for use in the treatment and management of inflammation in a subject in need of such treatment.

According to one or more embodiments, the HNK is selected from the group consisting of (2R,6R)-hydroxynorketamine, (2S,6S)-hydroxynorketamine, (2S,6R)-hydroxynorketamine, (2R,6S)-hydroxynorketamine, a salt thereof, or a combination thereof.

According to one or more embodiments, the HNK is (2R,6R)-hydroxynorketamine or a salt thereof. According to one or more embodiments, the HNK is (2S,6S)-hydroxynorketamine or a salt thereof. According to one or more embodiments, the HNK is (2S,6R)-hydroxynorketamine or a salt thereof. According to one or more embodiments, the HNK is (2R,6S)-hydroxynorketamine or a salt thereof. According to one or more embodiments, the HNK is a combination of (2R,6R)-hydroxynorketamine or a salt thereof, and (2S,6S)-hydroxynorketamine or a salt thereof.

According to one or more embodiments, the pharmaceutical composition is prescribed for administration in a route selected from the group consisting of: oral, intravenous, intraperitoneal, intranasal, subcutaneous, sublingual, intrathecal, transdermal, buccal, vaginal, rectal, topical, ocular, otic, and a combination thereof.

According to one or more embodiments, the dosage of the HNK within the composition is within the range of from 0.01 mg to 5000 mg, from 1 mg to 5000 mg, from 1 mg to 1000 mg, from 1 mg to 500 mg, or from 10 mg to 200 mg.

According to one or more embodiments, the HNK is prescribed for a prolonged administration comprising a sustained release dosage form of the HNK, a prolonged administration time of the HNK, a repeated administration, or a combination thereof.

According to one or more embodiments, the method comprising an immediate release dosage form of the HNK.

According to one or more embodiments, the prolonged administration time comprises HNK administration to the patient as an infusion over a period of 10 minutes to 48 hour, or 10 minutes to 24 hours, or 30 minutes to 12 hours, or 30 minutes to 4 hours.

According to one or more embodiments, the inflammation is associated with proinflammatory agents' secretion from monocyte cells.

According to one or more embodiments, the proinflammatory agents are cytokines. According to one or more embodiments, the proinflammatory agents are cytokines selected from a group consisting of IL-6, TNF-α, IL-8, IL-1β, or a combination thereof. According to one or more embodiments, the proinflammatory agent is a prostaglandin. According to one or more embodiments, the proinflammatory agent is prostaglandin E2 (PGE2).\

According to one or more embodiments, the inflammation is caused by a pathogen, a trauma, a hazardous substance, or an autoimmune disease.

According to one or more embodiments, the inflammation is caused by a pathogen selected from the group consisting of a virus, a bacteria, a protozoa, a prion, a viroid, or a fungus. According to one or more embodiments, the pathogen is a virus. According to one or more embodiments, the virus is a SARS-CoV-2.

According to one or more embodiments, the inflammation is a chronic inflammation, or an acute inflammation. According to one or more embodiments, the acute inflammation is caused by trauma, a hazardous substance, a microbial invasion, hemophagocytic lymphohistiocytosis (HLH), macrophage activation syndrome, sepsis.

According to one or more embodiments, the chronic inflammation is caused by an autoimmune disease. According to one or more embodiments, the autoimmune disease is selected from inflammatory bowel disease, psoriasis, multiple sclerosis, arthritis, or Systemic Lupus Erythematosus.

According to one or more embodiments, the inflammation is associated with an increased release of one or more of a proinflammatory agent. According to one or more embodiments, the proinflammatory agent is selected from one or more cytokines, one or more chemokines, one or more prostaglandins or a combination thereof. According to one or more embodiments, the inflammation is associated with an increased release of one or more of a proinflammatory agent selected from IL-6, TNF-α, IL-1β, IL-8 and PGE2.

According to one or more embodiments, the inflammation is a hyperinflammation.

According to one or more embodiments, the subject is a mammal. According to one or more embodiments, the subject is a human.

According to yet another aspect, the present invention provides a method of inhibiting or attenuating proinflammatory agents secretion from immune cells, the method comprises contacting the immune cells with a composition comprising an effective amount of HNK, a pharmaceutically acceptable salt thereof, or a combination thereof, thereby inhibiting or attenuating proinflammatory agents secretion from the immune cells.

According to one or more embodiments, the proinflammatory agents are cytokines. According to one or more embodiments, the proinflammatory agents are cytokines selected from a group consisting of IL-6, TNF-α, IL-8, IL-1β, and a combination thereof. According to one or more embodiments, the proinflammatory agents are prostaglandins. According to one or more embodiments, the proinflammatory agent is PGE2.

According to one or more embodiments, the HNK is selected from the group consisting of (2R,6R)-hydroxynorketamine, (2S,6S)-hydroxynorketamine, (2S,6R)-hydroxynorketamine, (2R,6S)-hydroxynorketamine, a salt thereof, or a combination thereof.

According to one or more embodiments, the HNK is (2R,6R)-hydroxynorketamine or a salt thereof. According to one or more embodiments, the HNK is (2S,6S)-hydroxynorketamine or a salt thereof. According to one or more embodiments, the HNK is (2S,6R)-hydroxynorketamine or a salt thereof. According to one or more embodiments, the HNK is (2R,6S)-hydroxynorketamine or a salt thereof. According to one or more embodiments, the HNK is a combination of (2R,6R)-hydroxynorketamine or a salt thereof, and (2S,6S)-hydroxynorketamine or a salt thereof.

According to one or more embodiments, the method comprising contacting the immune cells with the HNK for at least about 30 minutes. According to one or more embodiments, the method comprising contacting the immune cells with the HNK for at least about 12 hours. According to one or more embodiments, the method comprising contacting the immune cells with the HNK for at least about 24 hours. According to one or more embodiments, the method comprising contacting the immune cells with the HNK for at least about 48 hours.

According to one or more embodiments, the concentration of the HNK is at least about 0.1 μM. According to one or more embodiments, the concentration of the HNK is at least about 10 μM. According to one or more embodiments, the concentration of the HNK is at least about 50 μM. According to one or more embodiments, the concentration of the HNK is at least about 100 μM. According to one or more embodiments, the concentration of the HNK is at least about 150 μM. According to one or more embodiments, the concentration of the HNK is at least about 200 μM. According to one or more embodiments, the concentration of the HNK is at least about 250 μM.

According to one or more embodiments, the immune cells are monocytes or macrophages. According to one or more embodiments, the immune cells are human primary monocytes.

According to one or more embodiments, the immune cells are stimulated for enhanced proinflammatory agents secretion by a pathogen or a portion thereof selected from the group consisting of a virus, a bacteria, a protozoa, a prion, a viroid, or a fungus. According to one or more embodiments, the pathogen is a virus. According to one or more embodiments, the virus is a SARS-CoV-2.

According to one or more embodiments, the immune cells are stimulated for enhanced proinflammatory agents secretion by trauma, a hazardous substance, a microbial invasion, hemophagocytic lymphohistiocytosis (HLH), macrophage activation syndrome, or sepsis.

According to one or more embodiments, the immune cells are stimulated for enhanced proinflammatory agents secretion by an autoimmune disease. According to one or more embodiments, the autoimmune disease is selected from inflammatory bowel disease, psoriasis, multiple sclerosis, arthritis, or Systemic Lupus Erythematosus.

According to one or more embodiments, the HNK attenuates proinflammatory agents secretion from stimulated immune cells by at least about 20% compared to immune cells not subjected to treatment with HNK.

According to one or more embodiments, the present invention provides a method of treating inflammation/hyperinflammation, in accordance with the disclosure herein above. According to one or more embodiments, the present invention provides a pharmaceutical composition for use in the treatment of inflammation/hyperinflammation, in accordance with the disclosure herein above.

According to one or more embodiments, the present invention provides a pharmaceutical composition for use in the treatment of a viral/bacterial, or any pathogenic infection, in accordance with the disclosure herein above. According to one or more embodiments, the present invention provides a method of treating a viral and/or any infection, in accordance with the disclosure herein above. According to one or more embodiments, the present invention provides a method of treating SARS-CoV-2 infection, in accordance with the disclosure herein above. According to one or more embodiments, the present invention provides a method of treating a viral and/or any infection associated with inflammation/hyperinflammation, in accordance with the disclosure herein above. According to one or more embodiments, the present invention provides a method of treating SARS-CoV-2 infection associated with inflammation/hyperinflammation, in accordance with the disclosure herein above.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1—is a bar graph illustrating the 24 hours term effect of (2R,6R)-hydroxynorketamine hydrochloride (designated in the graph as (2R,6R)-HNK), (R,S)-norketamine (designated in the graph as (R,S)-NK), (R,S)-ketamine hydrochloride (designated in the graph as (R,S)-ket), and (R,S)-dehydronorketamine (designated in the graph as (R,S)-DHNK) at concentrations of 50 μM and 250 μM, and dexamethasone at a concentration of 1 μM, on the release of IL-6, TNF-α, IL-8 and Prostaglandin E2 (PGE2) from Lipopolysaccharides (LPS)-induced human primary monocytes (24 hours pre-treatment followed by 24 hours incubation with LPS, n=6, 3 donors).

FIG. 2—is a bar graph illustrating the 24 hours term effect of (2R,6R)-hydroxynorketamine hydrochloride (designated in the graph as (2R,6R)-HNK), (R,S)-norketamine (designated in the graph as (R,S)-NK), (R,S)-ketamine hydrochloride (designated in the graph as (R,S)-ket), and (R,S)-dehydronorketamine (designated in the graph as (R,S)-DHNK) at concentrations of 50 μM and 250 μM, and dexamethasone at a concentration of 1 μM on the viability of human primary monocytes (24 hours pre-treatment, followed by 24 hours incubation with LPS, n=6, 3 donors). Treatment with sodium fluoride (NaF) at μg/ml was used as control.

FIG. 3—is a bar graph illustrating the 48 hours term effect of (2R,6R)-hydroxynorketamine hydrochloride (designated in the graph as (2R,6R)-HNK), (R,S)-norketamine (designated in the graph as (R,S)-NK), (R,S)-ketamine hydrochloride (designated in the graph as (R,S)-ket), and (R,S)-dehydronorketamine (designated in the graph as (R,S)-DHNK) at concentrations of 50 μM and 250 μM, and dexamethasone at a concentration of 1 μM on the release of IL-6, TNF-α, IL-8, and PGE2 from LPS-induced human primary monocytes (48 hours pre-treatment followed by 24 hours incubation with LPS, n=6, 3 donors).

FIG. 4—is a bar graph illustrating the 48 hours term effect of (2R,6R)-hydroxynorketamine hydrochloride (designated in the graph as (2R,6R)-HNK), (R,S)-norketamine (designated in the graph as (R,S)-NK), (R,S)-ketamine hydrochloride (designated in the graph as (R,S)-ket), and (R,S)-dehydronorketamine (designated in the graph as (R,S)-DHNK) at concentrations of 50 μM and 250 μM, and dexamethasone at a concentration of 1 μM on the viability of human primary monocytes (48 hours pre-treatment followed by 24 hours incubation with LPS, n=6, 3 donors). Treatment with sodium fluoride (NaF) at μg/ml was used as control.

FIG. 5—is a bar graph illustrating the effect of (R,S)-ketamine hydrochloride at increasing concentrations (0.1 μM-250 μM) on the release of IL-6, IL-10, TNF-α, and IL-8 from LPS-induced human primary monocytes (30 min pre-treatment with (R,S)-ketamine hydrochloride, followed by 24 hours with LPS, n=6, 3 donors).

FIG. 6—is a bar graph illustrating the effect of (2R,6R)-hydroxynorketamine hydrochloride at increasing concentrations (0.1 μM-250 μM) on the release of IL-6, IL-1β, TNF-α, and IL-8 from LPS-induced human primary monocytes (30 minutes pre-treatment with (2R,6R)-hydroxynorketamine hydrochloride followed by 24 hours incubation with LPS, n=6, 3 donors).

FIG. 7—is a bar graph illustrating the effect of (2S,6S)-hydroxynorketamine hydrochloride at increasing concentrations (0.1 μM-250 μM) on the release of IL-6, IL-1β, TNF-α, and IL-8 from LPS-induced human primary monocytes (30 minutes pre-treatment with (2S,6S)-hydroxynorketamine hydrochloride followed by 24 hours incubation with LPS, n=6, 3 donors).

FIG. 8—is a bar graph illustrating the effect of (2R,6R)-hydroxynorketamine hydrochloride (designated in the graph as (2R,6R)-HNK), (2S,6S)-hydroxynorketamine hydrochloride (designated in the graph as (2S,6S)-HNK), and (R,S)-ketamine hydrochloride (designated in the graph as (R,S)-ket) at concentrations of 50 μM and 250 μM on the release of IL-6, IL-1β, TNF-α, and IL-8 from LPS-induced human primary monocytes (30 min pre-treatment followed by 24 h incubation with LPS, n=6, 3 donors).

FIG. 9—is a bar graph illustrating the 24 hours term effect of (2R,6R)-hydroxynorketamine hydrochloride (designated in the graph as (2R,6R)-HNK), (2S,6S)-hydroxynorketamine hydrochloride (designated in the graph as (2S,6S)-HNK), and (R,S)-ketamine hydrochloride (designated in the graph as (R,S)-ket) at concentrations of 50 μM and 250 μM on the release of IL-6, TNF-α, and IL-8 from LPS-induced human primary monocytes (24 hours pre-treatment, followed by 24 h incubation with LPS, n=6, 3 donors).

FIG. 10—is a bar graph illustrating the 48 hours term effect of (2R,6R)-hydroxynorketamine hydrochloride (designated in the graph as (2R,6R)-HNK), (2S,6S)-hydroxynorketamine hydrochloride (designated in the graph as (2S,6S)-HNK), and (R,S)-ketamine hydrochloride (designated in the graph as (R,S)-ket) at concentrations of 50 μM and 250 μM on the release of IL-6, TNF-α, and IL-8 from LPS-induced human primary monocytes (48 hours pre-treatment, followed by 24 h incubation with LPS, n=6, 3 donors).

### DETAILED DESCRIPTION OF THE INVENTION

The present invention pertains to, in some embodiments, methods and compositions for the treatment and management of inflammation comprising administering to a subject in need of such treatment a composition comprising hydroxynorketamine (HNK). The present invention further pertains to methods and compositions for inhibiting or attenuating proinflammatory agents' secretion, e.g., cytokines, from immune cells. Particularly, the invention pertains to methods of inhibiting TNF-alpha (TNF-α), IL-6, IL-8, PGE2 and/or IL-1beta (IL-1β) secretion from monocytes, the method comprising contacting the monocyte cells with hydroxynorketamine (HNK).

Ketamine has been demonstrated previously to possess anti-neuroinflammatory activity. Nevertheless, the use of this agent is limited due to accompanying side effects, such as dissociation, and addictive properties. The herein inventors surprisingly revealed that various ketamine metabolites act differently during inflammation. Specifically, (R,S)-norketamine (NK) induces the secretion of the proinflammatory agent PGE2, and dehydronorketamine (DHNK) seems to be toxic to monocytes. Conversely, hydroxynorketamine (HNK) was found to be effective in inhibiting proinflammatory agents' secretion from monocyte cells and did not cause monocytes mortality. (2R,6R)-HNK lacks psychotomimetic effects, locomotor effects, discoordination, and addictive potential. Thus, the present invention suggests the use of HNK in the treatment and management of inflammation.

As illustrated in the Examples section that follows, hydroxynorketamine (HNK), or 6-hydroxynorketamine, or 2-6-hydroxynorketamine (2,6-HNK) is herein demonstrated for the first time to possess anti-inflammatory properties. HNK has the chemical formula; C₁₂H₁₄ClNO₂ and is also known by the following synonyms: 2-Amino-2-(2-chlorophenyl)-6-hydroxycyclohexan-1-one, (2R,6R)-2-amino-2-(2-chlorophenyl)-6-hydroxycyclohexanone, HNK, (2R,6R)-2-amino-2-(2-chlorophenyl)-6-hydroxycyclohexan-1-one, (2R,6R)-HNK, (2R,6R)-hydroxynorketamine, 6-hydroxynorketamine (+−)-isomer, 6-hydroxynorketamine (2S-cis)-isomer, and (2S,65)-2-amino-2-(2-chlorophenyl)-6-hydroxycyclohexanone. Hydroxynorketamine has CAS registry #: 81395-70-2, (2R,6R)-HNK CAS #143020-69-9, (2S,6S)-HNK CAS #143020-70-2.

HNK may be (2R,6R)-hydroxynorketamine, (2S,6S)-hydroxynorketamine, (2S, 6R)-hydroxynorketamine, (2R, 6S)-hydroxynorketamine, salts thereof, or a combination thereof. Further contemplated are stereoisomers of hydroxynorketamine, and analogues thereof.

It is to be noted that the HNK may be essentially free, substantially free, or free of ketamine or a metabolite thereof other than HNK, i.e., the HNK is to be administered rather than ketamine, which would then generate HNK by its metabolism.

Thus, an aspect of the invention pertains to a method of inhibiting or attenuating proinflammatory agents (e.g., cytokines and/or chemokines) secretion from immune cells, the method comprises contacting the immune cells with a composition comprising hydroxynorketamine (HNK), thereby inhibiting or attenuating proinflammatory agents secretion from the immune cells.

As used herein the terms “inhibiting or attenuating” includes but is not limited to any one or more of the following: abrogating, ameliorating, blocking, suppressing, reducing, delaying, halting, alleviating or preventing the proinflammatory agents (such as, cytokines and/or chemokines, and/or prostaglandins) secretion from cells.

As used herein the terms “inhibiting” or “attenuating” includes contacting cells with HNK for a certain time period which may range from a few minutes and up to a few hours.

In some embodiments, contacting cells with HNK is conducted for at least about 5 minutes (min) and up to about 72 hours (h). For example, at least about 10 min and up to about 72 h, at least about 20 min and up to about 72 h, or at least about 30 min and up to about 72 hours. For example, the cells are exposed to the HNK for at least about 10 minutes, at least about 20 minutes, at least about 30 minutes, at least about 1 hour, at least about 2 hours, at least about 5 hours, at least about 10 hours, at least about 12 hours, at least about 24 hours, or at least about 48 hours. Each possibility represents a separate embodiment of the invention.

As used herein the term “proinflammatory agent” refers to a signaling molecule (such as cytokines, chemokines, and prostaglandins) that is secreted from immune cells, like helper T cells (Th), monocytes, macrophages, and certain other cell types and promote inflammation. Examples of cytokines include interleukins and interferons which are involved in regulating the immune system's response to inflammation and infection.

As used herein the term “chemokines” refers to a family of small cytokines, or signaling proteins secreted by cells. Chemokines are chemotactic cytokines. Their name is derived from their ability to induce directed chemotaxis in nearby responsive cells. Cytokine proteins are classified as chemokines according to behavior and structural characteristics. In addition to being known for mediating chemotaxis, chemokines are all approximately 8-10 kilodaltons in mass and have four cysteine residues in conserved locations that are key to forming their 3-dimensional shape.

The proinflammatory cytokines secretion from monocyte cells exemplified to be inhibited by HNK, include, Tumor necrosis factor-α (TNF-α, or TNF-α), Interleukin 8 (IL-8), Interleukin 6 (IL-6), and Interleukin 1β (IL-1β, or IL-1beta). Another proinflammatory agent exemplified to be inhibited by HNK, include, Prostaglandin (PGE2). Nevertheless, secretions of additional proinflammatory agents from immune cells are contemplated. Such cytokines include, without limitation, interleukin-1 (IL-1), interleukin-12 (IL-12), interleukin-18 (IL-18), interferon gamma (IFNγ), and granulocyte-macrophage colony stimulating factor (GM-CSF).

As used herein the term “immune cells” refers to a cell that is part of the immune system and helps the body fight infections and other diseases Immune cells develop from stem cells in the bone marrow and become different types of white blood cells Immune cells include neutrophils, eosinophils, basophils, mast cells, monocytes, macrophages, dendritic cells, natural killer cells, and lymphocytes (B cells and T cells). Additional cell types involved in the immune system may be contemplated, such as astrocytes that are crucial regulators of innate and adaptive immune responses in the injured central nervous system. In one embodiment, the immune cells are monocytes. In one embodiment, the immune cells are macrophages.

Optionally the immune cells are stimulated/triggered to secrete proinflammatory cytokines by a pathogen, a.k.a., an infectious agent (e.g., Lipopolysaccharides (LPS). The immune cells may be stimulated by a virus, a bacteria, a protozoan, a prion, a viroid, or a fungus. The immune cells may be stimulated, for example, due to an injury, a trauma, an autoimmune disease, a hazardous substance. Each possibility represents a separate embodiment of the invention.

Optionally, proinflammatory agent secretion from immune cells is inhibited by HNK by at least about 10%, 15%, 20% 30%, 40%, 50%, 60%, 70%, 80%, 90%, or any amount of reduction in between the specifically recited percentages, as compared to the levels without treatment with the HNK. Each possibility represents a separate embodiment of the invention.

For example, the secretion of TNF-α is inhibited by at least about 10% following treatment with HNK. In another example, the secretion of IL-6 is inhibited by at least about 10% following treatment with HNK.

Optionally, the treatment with HNK is conducted for at least about 30 minutes and the secretion of the proinflammatory agents that are inhibited from immune cells are IL-8, IL-6, IL-1β, and TNF-α. Optionally, the treatment with HNK is conducted for about 30 minutes and the secretion of the cytokines that are inhibited are IL-8, IL-6, IL-1β, and TNF-α. Optionally, the treatment with HNK is conducted for above 30 minutes, for example, at least about a few hours (e.g., 24 hours) and the secretion of the cytokines that are inhibited are IL-6, and TNF-α and further the secretion of PGE2 is inhibited.

The concentration of the HNK within the composition in which the immune cells are treated may be in a range of at least about 0.1 μM and up to about 1,000 μM (i.e., 1 mM). For example, the concentration of the HNK within the composition in which the immune cells are treated is at least about 1 μM, 10 μM, 50 μM, 100 μM, 150 μM, 200 μM, and 250 μM. Each possibility represents a separate embodiment of the invention.

For example, the secretion of TNF-α is inhibited by at least about 7%, or 10% following treatment with HNK at a concentration of 50 μM for 30 min. In another example, the secretion of IL-6 is inhibited by at least about 10%, or at least 15% following treatment with HNK at a concentration of 50 μM for 30 min. In another example, the secretion of IL-8 is inhibited by at least about 10% following treatment with HNK at a concentration of 50 μM for 30 min.

For example, the secretion of TNF-α is inhibited by at least about 20%, or 30% following treatment with HNK at a concentration of 250 μM for 30 min. In another example, the secretion of IL-6 is inhibited by at least about 15%, or at least 20% following treatment with HNK at a concentration of 250 μM for 30 min. In another example, the secretion of IL-8 is inhibited by at least about 15% or 20% following treatment with HNK at a concentration of 250 μM for 30 min. In another example, the secretion of IL-1β is inhibited by at least about 15% or 20% following treatment with HNK at a concentration of 250 μM for 30 min.

For example, the secretion of TNF-α is inhibited by at least about 10% following treatment with HNK at a concentration of 50 μM for 24 hours. In another example, the secretion of IL-6 is inhibited by at least about 10% following treatment with HNK at a concentration of 50 μM for 24 hours. In another example, the secretion of IL-8 is not inhibited following treatment with HNK at a concentration of 50 μM for 24 hours.

For example, the secretion of TNF-α is inhibited by at least about 30% or 40% following treatment with HNK at a concentration of 250 μM for 24 hours. In another example, the secretion of IL-6 is inhibited by at least about 10%, or at least 20% following treatment with HNK at a concentration of 250 μM for 24 hours. In another example, the secretion of IL-8 is not inhibited following treatment with HNK at a concentration of 250 μM for 24 hours.

For example, the secretion of TNF-α is inhibited by at least about 20% following treatment with HNK at a concentration of 50 μM for 48 hours. In another example, the secretion of IL-6 is inhibited by at least about 20% following treatment with HNK at a concentration of 50 μM for 48 hours. In another example, the secretion of IL-8 is not inhibited following treatment with HNK at a concentration of 50 μM for 48 hours.

For example, the secretion of TNF-α is inhibited by at least about 30% or 40% following treatment with HNK at a concentration of 250 μM for 48 hours. In another example, the secretion of IL-6 is inhibited by at least about 20%, or at least 30% following treatment with HNK at a concentration of 250 μM for 48 hours. In another example, the secretion of IL-8 is not inhibited following treatment with HNK at a concentration of 250 μM for 48 hours. Another aspect of the present invention is directed to methods and compositions for treating inflammation in a subject, comprising administering a pharmaceutical composition comprising a therapeutically effective amount of HNK to the subject in need of such treatment.

As used herein the term “inflammation” refers to a biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair. Currently used therapeutic agents for inflammation include ibuprofen, antihistamines, steroids, and cortisones, but these drugs are associated with side effects such as temporary or simple symptoms, hypersensitivity reactions, and immune system deterioration.

There is thus a need for safe, and effective anti-inflammatory drugs. The present invention pertains to the use of HNK for the treatment of inflammation.

The inflammation may be an acute inflammation, a chronic inflammation, or a delayed inflammatory response. Non-limited examples of conditions associated with an acute inflammatory response include, trauma, hazardous substances, microbial invasion, hemophagocytic lymphohistiocytosis (HLH), macrophage activation syndrome, sepsis and cytokine release syndrome in the setting of immunotherapy. Further, non-limited examples of conditions associated with chronic inflammation include autoimmune diseases (e.g., inflammatory bowel disease, psoriasis, rheumatoid arthritis, psoriatic arthritis, multiple sclerosis, arthritis, Systemic Lupus Erythematosus, etc.).

In an embodiment of the invention, the inflammation is a neuroinflammation. Optionally, the inflammation is associated with a neurological or a psychiatric disease, including, but not limited to, depression, Alzheimer's disease, or Amyotrophic lateral sclerosis (ALS). Optionally, the inflammation is not associated with a neurological or psychiatric disease, including, but not limited to, depression, Alzheimer's disease, or amyotrophic lateral sclerosis (ALS). In an embodiment of the invention, the inflammation is other than neuroinflammation. Optionally, the herein method of treatment is other than treating bipolar depression, major depressive disorder, schizophrenia, Alzheimer's dementia, amyotrophic lateral sclerosis, complex regional pain syndrome (CRPS), chronic pain, or neuropathic pain. Each possibility represents a separate embodiment of the invention. The inflammation may involve a pathogen infection. The pathogen may be a virus, bacteria, protozoa, a prion, a viroid, a helminth, or a fungus. In an embodiment of the invention, the inflammation is associated with increased secretion of proinflammatory agents. In an embodiment of the invention, the inflammation is associated with an increased release of one or more of IL-6, TNF-α, IL-1β, IL-8 and PGE2. Each possibility presents a separate embodiment of the invention.

Optionally, the inflammation is hyper inflammation. Thus, in an embodiment of the invention, the HNK is effective in attenuating or suppressing hyperinflammation in a subject.

As used herein the term “Hyperinflammation” refers to syndromes, disorders or conditions caused by severe and uncontrolled immune cell activation and hypercytokinemia. The clinical presentation inter alia includes, unremitting fever, splenomegaly, coagulopathy, hepatitis, cytopenia and, if unrestrained, multi-organ failure and death.

As used herein the term “Cytokine Storm” encompasses a multi-factorial hyperinflammation associated inter alia with increased expression of one or more of interleukins (IL), chemokines, granulocyte colony-stimulating stimulating factors (G-CSF), and tumor necrosis factor (TNF)-1α.

Various physiological conditions may be associated with hyperinflammation. Exemplary conditions include, but not limited to, Familial Hemophagocytic Lymphohistiocytosis, Immunodeficiency-Associated Hyperinflammatory syndrome, Macrophage Activation Syndrome, Infection-Associated Hyperinflammatory Syndrome, and Malignancy-Associated Hyperinflammatory Syndrome.

An aspect of the present invention is directed to methods and compositions for treating a subject afflicted with inflammation. An aspect of the present invention is directed to methods and compositions for treating a subject afflicted with an infectious disease. An aspect of the present invention is directed to methods and compositions for treating inflammation caused by an infectious disease in a subject. As aspect of the invention pertain to methods and compositions for treating inflammation in a subject, comprising administering to the subject a pharmaceutical composition comprising a therapeutic effective amount of HNK. An aspect of the present invention pertains to methods and compositions for treating a viral disease associated with inflammation in a subject, comprising administering to the subject a pharmaceutical composition comprising a therapeutic effective amount of HNK. An aspect of the invention provides a method of treating a viral infection in a subject in need thereof, the method comprising administering to the subject a therapeutic effective amount of a pharmaceutical composition comprising HNK.

In an embodiment of the invention, the HNK, is effective in the treatment of subjects afflicted with a corona virus. In an embodiment of the invention, the HNK, is effective in the treatment and management of inflammation in subjects afflicted with a corona virus.

As used herein the term “coronavirus” refers to a family of various viruses, including without limitation, SARS coronavirus (SARS-CoV), MERS coronavirus (MERS-CoV), and SARS-CoV-2 (that causes COVID-19). Each possibility represents a separate embodiment of the invention. In an embodiment of the invention, the HNK is effective in the treatment of subjects afflicted with a SARS-CoV-2.

In an embodiment of the invention, the method may comprise a step of identifying patient anxiety and depression associated with hospitalization and social distancing due to SARS-CoV-2 infection using the Hospital Anxiety and Depression Scale (HADS) and treating, in addition to the SARS-CoV-2-associated inflammation, the anxiety and depression by administering a therapeutic effective amount of HNK.

Accumulating evidence suggest that some patients with severe COVID-19 might experience hyperinflammation expressed by robust cytokine release (a.k.a., cytokine storm syndrome or secondary haemophagocytic lymphohistiocytosis (sHLH). Thus, in an embodiment of the invention, the HNK is effective in attenuating or suppressing hyperinflammation associated with a viral infection. In an embodiment of the invention, the HNK is effective in attenuating or suppressing hyperinflammation associated with a corona virus infection. In an embodiment of the invention, the HNK is effective in attenuating or suppressing hyperinflammation associated with SARS-CoV-2 infection.

In an embodiment of the invention, the HNK is effective in attenuating or suppressing cytokine storm syndrome or secondary haemophagocytic lymphohistiocytosis (sHLH).

In an embodiment of the invention, the method comprising identifying hyperinflammation in the subject and treating the hyperinflammation comprising administering to the subject a pharmaceutical composition comprising a therapeutic effective amount of HNK.

Identifying hyperinflammation may include assessment of inflammatory markers in a biological sample of a subject, including assessment of increased levels of inflammatory proteins, such as cytokines and chemokines (e.g., IL-1β, IL-6, IL-8, tumor necrosis factor-1a), C reactive protein (CRP), growth factors (e.g., granulocyte colony-stimulating stimulating factors (G-CSF) and ferritin.

Hyperinflammation can be diagnosed using laboratory methodologies. Non limited examples of suitable laboratory methodologies include ferritin assessment, platelet count assessment, erythrocyte sedimentation rate determination, to identify the subgroup of patients for whom immunosuppression treatment could be effective.

Optionality, the HNK will be administered as a single agent or in combination with other immunosuppressive drugs including, for example: beta2-adrenergic receptor agonists (e.g., metaproterenol, terbutaline, salbutamol, salmeterol, (R,R′)-4′-methoxy-1-naphthylfenoterol); GPR55 receptor antagonists (e.g., cannabidiol, (R,R′)-4′-methoxy-1-naphthylfenoterol), High-mobility group box 1 (HMGB1) protein inhibitors (e.g., heparin and ascorbic acid); Janus kinase (JAK) inhibitors (e.g., tofacitinib, bracicitinib).

Optionally, the treatment with HNK may additionally include treatment with immunosuppressive agents, steroids, intravenous immunoglobulin, selective cytokine blockade (e.g., anakinra or tocilizumab) and JAK inhibition.

Optionally, the HNK is used in combination with antimicrobials agent, such as antiviral/antiretroviral agents, and antibacterial agents, to thereby manage the symptoms of infections, superinfections and hyperinflammation.

Exemplary antiviral/antiretroviral agents include, without limitation, agents that inhibit viral replication cycle (e.g., agents that inhibit viral DNA polymerase), agents that inhibit viral cell penetration and/or protein synthesis. In an embodiment of the invention, the antiviral/retroviral agent is effective in treating coronaviruses infections (e.g., anti-coronavirus antibodies, stem cells-based treatments etc.).

Antibacterial agents are typically capable of reducing the metabolic activity of bacteria such that their pathogenic effect in the biological environment will be minimized or eliminated. Exemplary antibiotics include, without limitation, β-Lactam antibiotics (penicillins, cephalosporins, carbapenems, monobactams), Tetracyclines (e.g., doxycycline, minocycline), macrolide antibiotics, aminoglycosides, peptide antibiotics, lincosamides, and streptogramins

Administration of HNK may be before, at the onset of, or following inflammation/hyperinflammation detection in the subject.

The HNK may be administered via an oral (per os; p.o.), an intravenous (i.v.), a sublingual (s.l.), an intraperitoneal (i.p.), and an intranasal (i.n.) administration for a direct pulmonary dosing. Nevertheless, alternative administration routes are further contemplated and may be applicable (e.g., rectal, intramuscular, subcutaneous (s.c.), transdermal, intraocular, intrathecal, buccal, vaginal).

The HNK may be administered to the patient at a frequency of: once per day, twice per day, three times per day, or four times per day. The frequency of administration may be every few days (i.e., every 2, 3, 4, 5, 6, or 7 days, etc.), once a week, or once every few weeks (i.e., once every 2, 3, or 4 weeks, etc.).

The route of HNK administration may be as an immediate release form, a timed/delayed-release form, or a control/slow/sustained-release form, or a combination thereof. Each possibility represents a separate embodiment of the invention.

In one embodiment, the HNK is administered in a prolonged treatment regime or procedure, i.e., the vehicle of HNK is a sustained release vehicle, or the administration itself is prolonged or repeated.

Optionally, the HNK is administered as an immediate release form to treat conditions associated with increased IL-8, IL-6, TNF-α, and/or IL-1β release from immune cells.

Optionally, the HNK is administered as an immediate release form to treat conditions associated with increased IL-8 release from immune cells. Exemplary conditions associated with an increased IL-8 release include, for example, chronic inflammatory conditions such as psoriasis, acute respiratory distress syndrome (ARDS), chronic obstructive pulmonary disease (COPD), rheumatoid arthritis (RA), or increased IL-8 expression in cancer.

Optionally, the HNK is administered as a sustained release dosage form or provided for prolonged time to treat conditions associated with increased IL-6, and/or TNF-α and/or PGE2.

As used herein the term “sustained release dosage form” refers to a dosage form designed to release (liberate) a drug at a predetermined rate in order to maintain a constant drug concentration for a specific period of time. Exemplary sustained release dosage forms include, without limitation, polymers, various coating substances, semipermeable membranes, liposomes, hydrogels, and the like.

Optionally, the prolonged administration time comprises HNK administration to the patient as an infusion over a period of 5 minutes to 48 hours, 10 minutes to 48 hour, or 10 minutes to 24 hours, or 10 minutes to 12 hours, or 10 minutes to 6 hours, or 10 minutes to 4 hours, or 10 minutes to 2 hours, or 30 minutes to 48 hours, or 30 minutes to 24 hours, or 30 minutes to 12 hours, or 30 minutes to 6 hours, or 30 minutes to 4 hours. Each possibility represents a separate embodiment of the invention.

Optionally, the HNK administration is for at least about 5 minutes, at least about 10 minutes, at least about 20 minutes, at least about 30 minutes, at least about 40 minutes, at least about 50 minutes, at least about 60 minutes, at least about 2 hours, at least about 4 hours, at least about 6 hours, at least about 8 hours, at least about 10 hours, at least about 12 hours, at least about 24 hours, or at least about 48 hours. Each possibility represents a separate embodiment of the invention.

The term “therapeutically effective amount” or “effective amount” means an amount effective, when administered to a human or non-human patient, to provide any therapeutic benefit. A therapeutic benefit may be an amelioration of symptoms, e.g., an amount effective to decrease the symptoms of inflammation. A therapeutically effective amount of a compound is also an amount sufficient to provide a significant positive effect on any indicia of a disease, disorder, or condition e.g., an amount sufficient to significantly reduce the severity of inflammation. A significant effect on an indicia of a disorder or condition includes a statistically significant in a standard parametric test of statistical significance such as Student's T-test, where p<0.05; though the effect need not be significant in some embodiments.

The amount of HNK for administration to the subject in a dosage form may be within the range of from 0.01 mg to 5000 mg. For example, from 0.1 mg to 5000 mg, from 1 mg to 5000 mg, from 1 mg to 1000 mg, from 1 mg to 500 mg, or from 10 mg to 200 mg. For example, the HNK is provided in an amount within the range of about 0.05-1 mg/kg. For example, about 0.1-0.5 mg/kg, about 0.1-0.4 mg/kg, 0.1-0.3 mg/kg, or 0.15-0.25 mg/kg. The amount of HNK for administration to the subject in a dosage form may be at least about 0.01 mg, 0.1 mg, 1 mg, 10 mg, or above. Each possibility represents a separate embodiment of the invention.

The concentration of the HNK within the composition for administration may be in a range of at least about 0.1 μM and up to about 1000 μM (i.e., 1 mM). For example, the concentration of the HNK is at least about 1 μM, 10 μM, 50 μM, 100 μM, 150 μM, 200 μM, and 250 μM. Each possibility represents a separate embodiment of the invention.

As used herein the term “subject” means any human or non-human animal in need of medical treatment. In some embodiments, the subject is a mammal In some embodiments, the subject is a human.

As used herein the term “pharmaceutical compositions” refers to compositions comprising at least one active pharmaceutical agent, i.e., HNK as herein disclosed, and at least one other substance, such as a carrier, excipient, or diluent.

As used herein the term “prodrug” refers to a medication or compound that, after administration, is metabolized (i.e., converted within the body) into a pharmacologically active drug. Inactive prodrugs are pharmacologically inactive medications that are metabolized into an active form within the body.

The term “pharmaceutically acceptable carrier” applied to pharmaceutical compositions of the invention refers to a diluent, excipient, or vehicle with which the herein HNK compound is administered. The carrier refers to an excipient that is useful in preparing a pharmaceutical composition that is generally safe, non-toxic, and neither biologically nor otherwise undesirable, and includes excipients that are acceptable for veterinary use as well as human pharmaceutical use.

By the term “carrier” it is meant to refer to any substance suitable as a vehicle for delivering of the HNK of the present invention to a suitable biological site or tissue. As such, carriers can act as a pharmaceutically acceptable excipient of the pharmaceutical composition of the present invention. Carriers of the present invention may include: (1) excipients or formularies that transport, but do not specifically target a molecule to a cell (referred to herein as non-targeting carriers); and (2) excipients or formularies that deliver a molecule to a specific site in a subject or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Pharmaceutical compositions of the present invention may be sterilized by conventional methods.

Suitable carriers may further include agents that are capable of delivering HNK to a target site in a subject. Examples of delivery vehicles include, but are not limited to, artificial and natural lipid-containing delivery vehicles. Natural lipid-containing delivery vehicles include cells and cellular membranes. Artificial lipid-containing delivery vehicles include liposomes and micelles.

As used herein “pharmaceutically acceptable salts” are derivatives of the disclosed compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts.

Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like, and combinations comprising one or more of the foregoing salts.

The terms “a” and “an” do not denote a limitation of quantity, but rather denote the presence of at least one of the referenced item.

The term “stereoisomers” are compounds, which have identical chemical constitution, but differ with regard to the arrangement of the atoms or groups in space.

The term “enantiomers” refer to two stereoisomers of a compound, which are nonsuperimposable mirror images of one another. A 50:50 mixture of enantiomers is referred to as a racemic mixture or a racemate, which may occur where there has been no stereoselection or stereo specificity in a chemical reaction or process.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

An “active agent” means any compound, element, or mixture that when administered to a patient alone or in combination with another agent confers, directly or indirectly, a physiological effect on the patient. When the active agent is a compound, salts, solvates (including hydrates) of the free compound or salt, crystalline and non-crystalline forms, as well as various polymorphs of the compound are included. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms.

As used herein “administration” means dispensing a compound or composition containing the compound for use via any appropriate route, for example, oral administration, in either solid or liquid dosage form, inhalation, injection, suppository administration, or transdermal contact. “Administration” also includes applying a compound or composition containing the compound via any appropriate route such as via oral administration, in either solid or liquid dosage form, inhalation, injection, suppository administration, or transdermal contact. “Administration” does not include in vivo formation of the compound by a metabolic pathway in a person or animal who has consumed or been treated with ketamine or norketamine.

The term “consisting essentially of” means that the composition, method or structure may include additional ingredients, steps and/or parts, but only if the additional ingredients, steps and/or parts do not materially alter the basic and novel characteristics of the claimed composition, method or structure.

The term “consisting of” means including and limited to.

As used herein the term “about” refers to ±10% or ±5%.

The terms “comprise”, “comprising”, “includes”, “including”, “having” and their conjugates mean “including but not limited to”.

As use herein, the term “substantially free of” means that the composition comprises less than about 0.5% by weight, less than about 0.4% by weight, less than about 0.3% by weight, less than about 0.2% by weight, or less than about 0.1% by weight, of an ingredient unless specifically indicated otherwise.

As used herein, the term “essentially free of” means that the composition comprises less than about 0.05% by weight, less than about 0.01% by weight, or less than about 0.001% by weight of the ingredient, unless specifically indicated otherwise.

As used herein, the term “free of” means that the composition does not comprise the ingredient or comprises a trace amount of the ingredient, unless specifically indicated otherwise.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### EXAMPLES

The invention will be described in detail by way of specific examples. The following examples are offered for illustrative purposes and are not intended to limit the invention in any manner. Those of skill in the art will readily recognize a variety of non-critical parameters, which can be changed or modified to yield essentially the same results. In one or more embodiments, the amounts in the examples should be read with the prefix “about”.

### Materials

The following agents were used in the herein tests. (2S,6S)-Hydroxynorketamine hydrochloride; (2R,6R)-hydroxynorketamine hydrochloride; (R,S)-ketamine hydrochloride; (R,S)-norketamine (NK); (R,S)-dehydronorketamine (DHNK); and dexamethasone (Sigma-Merck, Taufkirchen, Germany)

### Methods

Measurement of cytokines release from primary human monocytes—human primary monocytes were isolated (enriched) from buffy coats of healthy human blood donors. Cells were seeded in 24-well-plates for ELISA experiments. The results were normalized to LPS, provided as 100%, and presented as percentage of change cytokine levels. The various respective statistical significances were calculated by T-Test.

Viability of human primary monocytes—viable human primary monocyte cells were counted using Alamar Blue. Treatment with toxic amounts of sodium fluoride (NaF at 250 μg/ml) was used as control.

### Example 1—The Effect Ketamine and Metabolites thereof on Proinflammatory Agents Release from LPS Stimulated Human Primary Monocytes

The following study evaluated the effect of ketamine and ketamine metabolites on the release of the cytokines IL-6, IL-8, and TNF-α and on Prostaglandin E2 (PGE2) from LPS-induced primary human monocytes. To this end, human primary monocytes were incubated for 24 hours and 48 hours with (R,S)-ketamine, (2R,6R)-hydroxynorketamine, (R,S)-norketamine, and (R,S)-dehydronorketamine at concentrations of 50 μM and 250 μM. LPS alone and treatment with dexamethasone (1 μM) were used as controls. Monocyte cells' viability was measured using Alamar blue to evaluate toxicity of the agents.

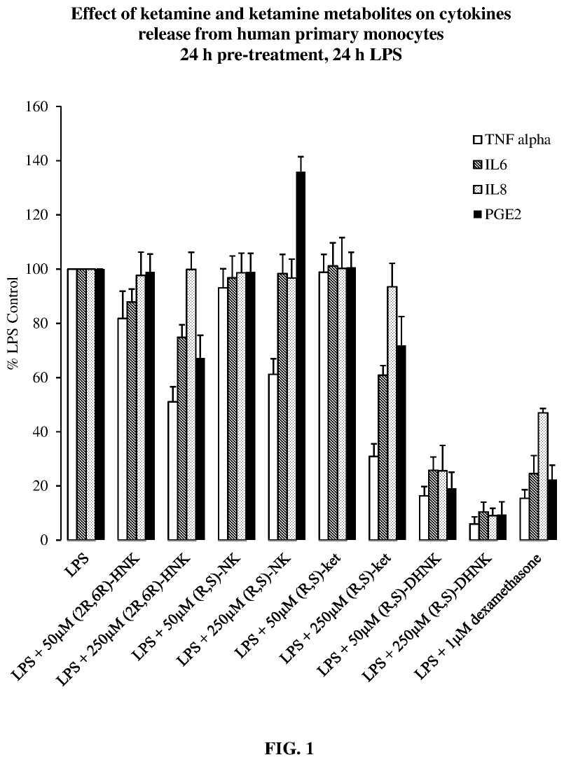

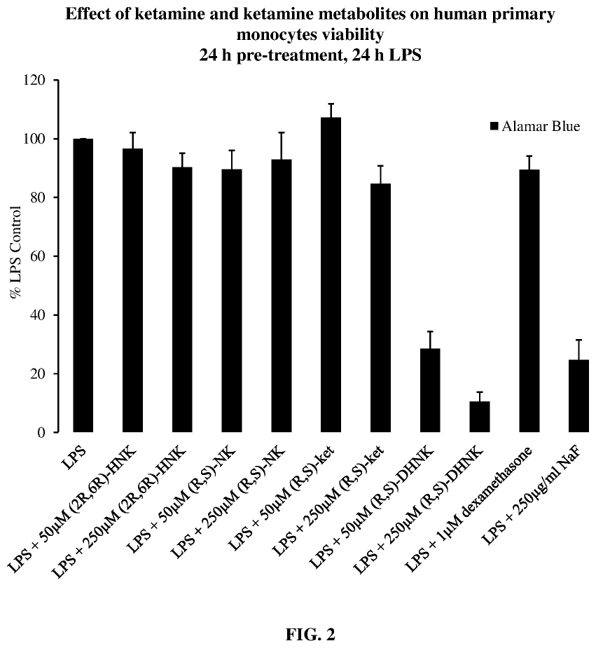

FIG. 1 illustrates the effect of the above agents following 24 hours pre-treatment on the cytokines IL-6, IL-8, and TNF-α and PGE2 release from human primary monocytes. FIG. 2 illustrates the viability of the cells following treatments. As can be seen in FIG. 1, treatment with (R,S)-ketamine and its metabolite (2R,6R)-hydroxynorketamine illustrated inhibition in the secretion of TNF-α, IL-6, and PGE2 from the cells in a dose dependent manner, effecting higher inhibition of secretion levels at the higher dose of 250 μM, compared to the 50 μM treatment. (R,S)-Norketamine inhibited the secretion of TNF-α only at the higher dose tested and promoted the secretion of PGE2. IL-6 and IL-8 remained without any significant change in the secretion levels. Treatment with (R,S)-dehydronorketamine dramatically reduced all cytokines release and also PGE2 release, but was highly toxic to the cells, and induced a massive monocyte cells' death (FIG. 2). Without being bound by any theory or mechanism of action, it may be that the very low cytokine levels detected by the (R,S)-dehydronorketamine treatment represent cell death and not many cells secreting cytokines.

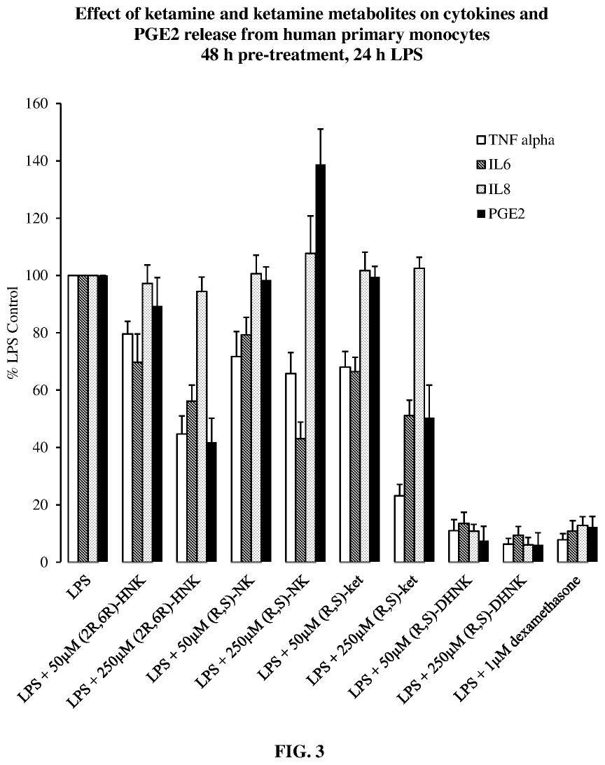

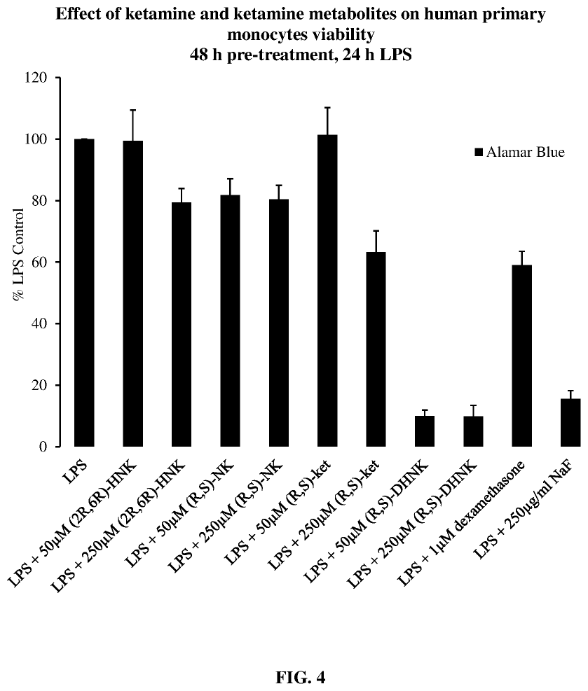

FIG. 3 illustrates the effect of the above agents following 48 hours pre-treatment on the cytokines IL-6, IL-8, and TNF-α and PGE2 release from human primary monocytes. FIG. 4 illustrates the viability of the treated cells following the 48 hours treatments. As can be seen in FIG. 3, treatment with (R,S)-ketamine and its metabolite (2R,6R)-hydroxynorketamine illustrated inhibition of TNF-α, IL-6, and PGE2 secretion from the cells in a dose dependent manner effecting higher inhibition of secretion levels at the higher dose of 250 μM, compared to the 50 μM treatment. (R,S)-Norketamine inhibited the secretion of TNF-α and IL-6 exhibiting a higher effect at the higher dose, but this agent promoted the secretion of the proinflammatory agent PGE2 at 250 μM. Treatment with (R,S)-dehydronorketamine dramatically reduced all cytokines release and also PGE2 and induced a massive cells' mortality, reflecting its toxic effect on the cells, (FIG. 4).

The results elucidate the anti-inflammatory potential of (2R,6R)-hydroxynorketamine in the inhibition of pro-inflammatory cytokines and PGE2 secretion from monocyte cells. The results further reveal that the various ketamine metabolites have different activity. The ketamine metabolite (R,S)-dehydronorketamine demonstrated cytotoxicity to the monocytes. Further, although (R,S)-norketamine inhibited the secretion of TNF-α, this agent induced PGE2 secretion which is a pro-inflammatory agent. In view of the above, (2R,6R)-hydroxynorketamine illustrated both anti-inflammatory properties and monocytes safety, suggesting the use of this agent in the treatment and management of inflammation. It is to be noted that ketamine itself, when administered in-vivo, is rapidly metabolized to its metabolites, so its in-vitro effect shown herein is not necessarily sustainable in-vivo. Namely, ketamine administration in-vivo is associated with its known side effect and may additionally illustrate toxicity toward monocytes which is mediated at least by its metabolite (R,S)-dehydronorketamine.

### Example 2—The Effect of Short Period Pre-Treatment with (R,S)-Ketamine Hydrochloride, and Ketamine Metabolites on Proinflammatory Cytokines Release in LPS Treated Human Primary Monocytes

The following study evaluated the short-term pre-treatment effect of (R,S)-ketamine hydrochloride, (2R,6R)-hydroxynorketamine hydrochloride, and (2S,6S)-hydroxynorketamine hydrochloride at increasing concentrations on cytokines release from LPS-induced primary human monocytes (pre-incubation time of 30 min before LPS treatment).

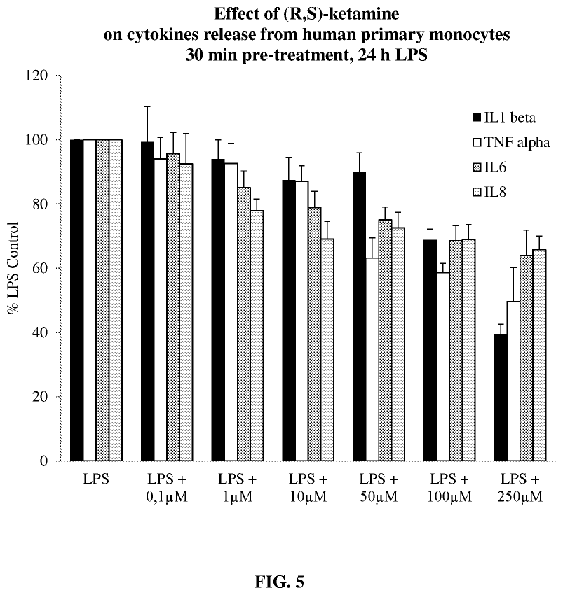

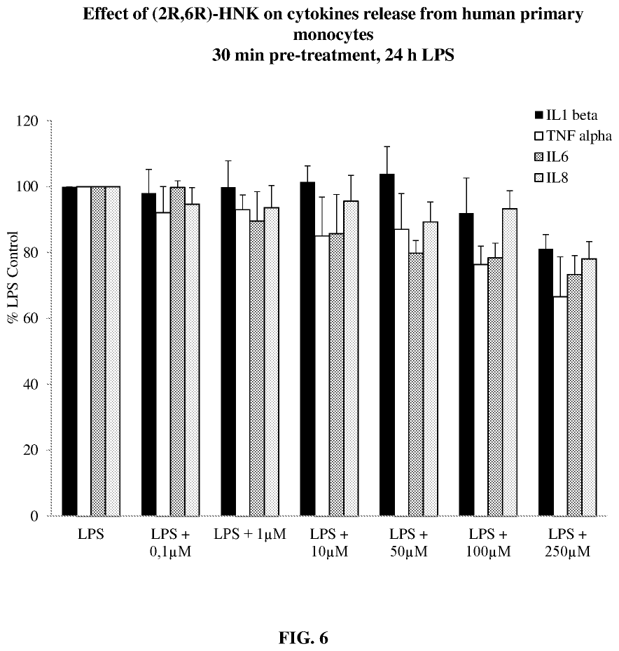

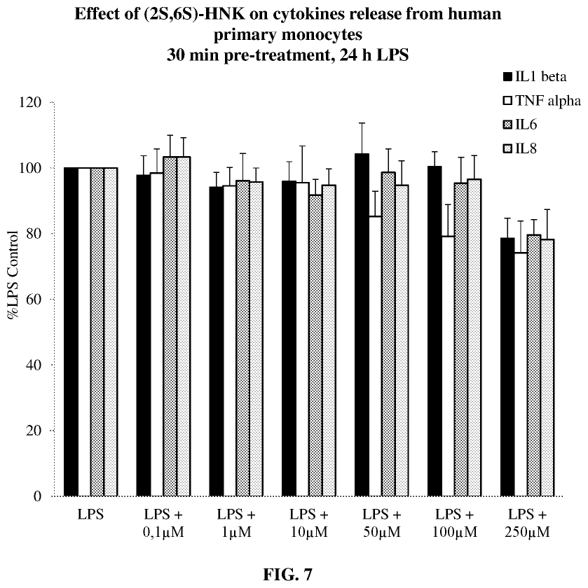

Human primary monocytes were incubated for 30 min with increasing doses of (R,S)-ketamine hydrochloride, (2R,6R)-hydroxynorketamine hydrochloride, and (2S,6S)-hydroxynorketamine hydrochloride (0.1 μM-250 μM). Cells were then stimulated for 24 hours with LPS. As can be seen in FIG. 5, (R,S)-ketamine hydrochloride exhibits a potent inhibitory effect on the secretion of all proinflammatory cytokines tested. (2R,6R)-Hydroxynorketamine hydrochloride and (2S,6S)-hydroxynorketamine hydrochloride also illustrated cytokines release decrease in a dose dependent manner (FIGS. 6 and 7, respectively). Dexamethasone was used as control and illustrated a highly potent inhibitory effect on the cytokines release demonstrating about 60% reduction effect at 10 μM (not shown).

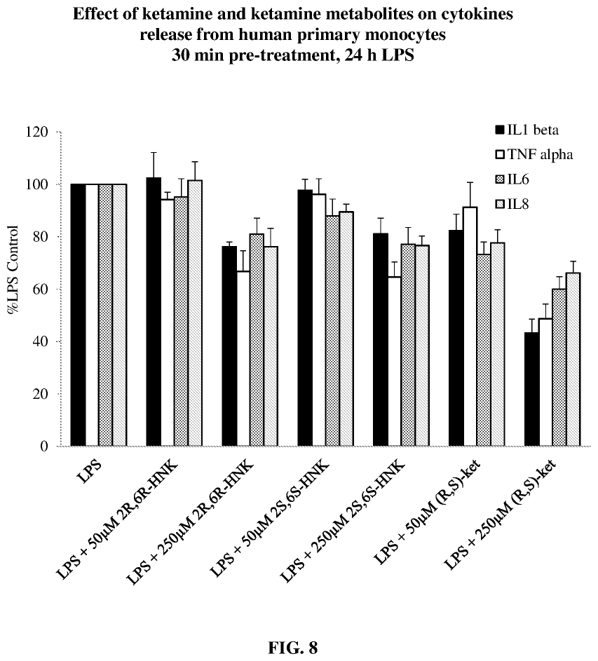

FIG. 8 illustrates the release of the cytokines IL-1β, TNF-α, IL6 and IL8 following 30 min time pre-treatment with (R,S)-ketamine hydrochloride, (2R,6R)-hydroxynorketamine hydrochloride, and (2S,6S)-hydroxynorketamine hydrochloride at 50 μM and 250 μM. As shown in FIG. 8, (R,S)-ketamine hydrochloride, (2R,6R)-hydroxynorketamine hydrochloride and (2S,6S)-hydroxynorketamine hydrochloride showed comparable data in the 30 min pre-stimulation approach at 50 μM and 250 μM relative to the results shown in FIGS. 5, 6 and 7 with only minor differences. (2R,6R)-Hydroxynorketamine hydrochloride and (2S,6S)-hydroxynorketamine hydrochloride slightly inhibited LPS-induced TNF-α release but also the other cytokines in the dose of 250 μM, approx. 30% inhibition for TNF-α and 20% inhibition for IL-6, IL-8, and IL-1β. (R,S)-ketamine hydrochloride was the most potent compound and prevented LPS-induced IL-6, IL-8, TNF-α, and IL-1β releases at a range of between 30-60% using the doses of 250 μM, with IL-1β being the most affected cytokine.

### Example 3—The Effect of 24 Hours Pre-Treatment with (R,S)-Ketamine Hydrochloride, (2R,6R)-Hydroxynorketamine Hydrochloride, and (2S,6S)-Hydroxynorketamine Hydrochloride on Cytokines Release in LPS Treated Human Primary Monocytes

The following study evaluated the long-term pre-treatment effect of (R,S)-ketamine hydrochloride, (2S,6S)-hydroxynorketamine hydrochloride and (2R,6R)-hydroxynorketamine hydrochloride at 50 μM and 250 μM on LPS-induced primary human monocytes with a pre-incubation time of 24 h followed by 24 h LPS treatment.

Primary human monocytes were pre-treated for 24 hours with (R,S)-ketamine hydrochloride, (2R,6R)-hydroxynorketamine hydrochloride, and (2S,6S)-hydroxynorketamine hydrochloride at 50 μM and 250 μM. Following the pre-treatment, LPS was added for additional period of 24 hours. The secretion of IL-6, TNF-α, and IL-8 was evaluated using an ELISA assay.

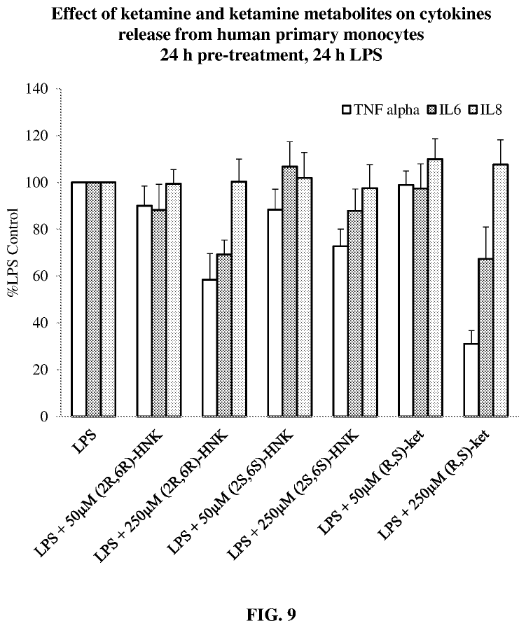

As shown in FIG. 9, (2R,6R)-hydroxynorketamine hydrochloride at the dose of 250 μM inhibited LPS-induced TNF-α and IL-6 release by approx. 40% and 35%, respectively, whereas IL-8 was not affected. (2S,6S)-hydroxynorketamine hydrochloride was slightly less effective and inhibited LPS-induced TNF-α release approx. 30% inhibition and IL-6 about 10%. LPS-induced IL-8 was not affected. Again, (R,S)-ketamine hydrochloride was the most potent compound and prevented LPS-induced TNF-α release about 70% in the dose of 250 μM and IL-6 about 30% without affecting IL-8.

The herein results, elucidate the higher effect of prolong treatment with HNK, especially of (2R,6R)-Hydroxynorketamine.

### Example 4—The Effect of 48 Hours Pre-Treatment with (R,S)-Ketamine Hydrochloride, (2R,6R)-Hydroxynorketamine Hydrochloride, and (2S,6S)-Hydroxynorketamine Hydrochloride on Cytokines Release in LPS Treated Human Primary Monocytes

The following study evaluated the long-term pre-treatment effect of (R,S)-ketamine hydrochloride, (2S,6S)-hydroxynorketamine hydrochloride, and (2R,6R)-hydroxynorketamine hydrochloride at 50 μM and 250 μM on LPS-induced primary human monocytes with a pre-incubation time of 48 h before 24 h LPS treatment.

Primary human monocytes were pre-treated for 48 hours with (R,S)-ketamine hydrochloride, (2R,6R)-hydroxynorketamine hydrochloride, and (2S,6S)-hydroxynorketamine hydrochloride at 50 μM and 250 μM. Following the pre-treatment, LPS was added for additional period of 24 hours. The secretion of IL-6, TNF-α, and IL-8 was evaluated using an ELISA assay.

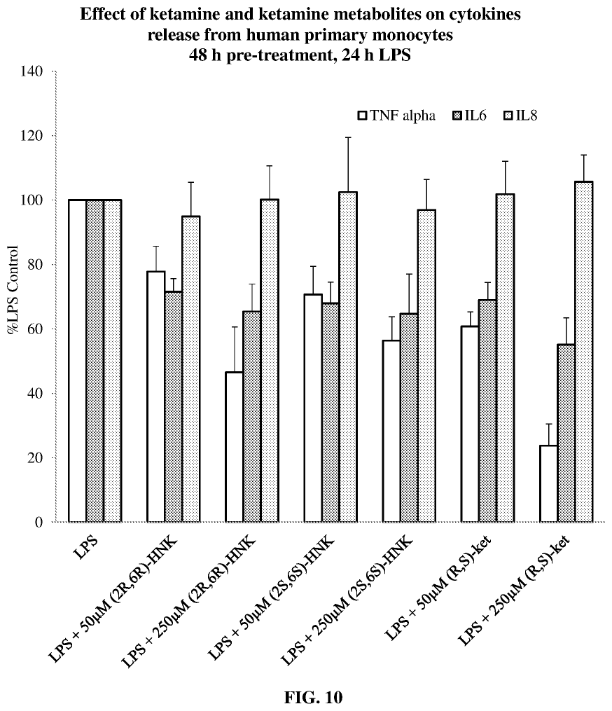

In the 48 h pre-treatment approach, all three compounds inhibited LPS-induced IL-6 and TNF-α in all doses tested. IL-6 release was prevented by approx. 30%-40% by all ketamines. (2R,6R)-Hydroxynorketamine hydrochloride inhibited LPS-induced TNF-α about 20% using the dose of 50 μM and approx. 55% in the dose of 250 μM. (2S,6S)-hydroxynorketamine hydrochloride showed inhibited LPS-induced TNF-α release about 30% in the dose of 50 μM and approx. 40% using the dose of 250 μM. (R,S)-ketamine hydrochloride was the most potent ketamine also after 48 h of pre-treatment and prevented LPS-induced TNF-α release about 40% using the dose of 50 μM and 75% using the dose of 250 μM. All ketamines did not affect LPS-mediated IL-8 release (FIG. 10).

In summary, at the 48 h pre-stimulation, (2R,6R)-hydroxynorketamine hydrochloride and (2S,6S)-hydroxynorketamine hydrochloride were more effective showing similar inhibition on LPS-induced IL-6 as (R,S)-ketamine hydrochloride and revealed potent inhibitory activities on TNF-α.

As illustrated herein, in the 24 h pre-treatment period there was no inhibition on IL-8 anymore. One major difference was also that LPS lost the capability to induce IL-1β after 24 h and also after 48 h showing only a marginal synthesis at the bottom of the detection limit of the ELISA (data not shown).

Thus, the long-term pre-stimulation for 48 h improved the anti-inflammatory activities of the ketamines on TNF-α and IL-6 but interestingly had no effect on IL-8. Thus, in one or more embodiments, the HNK may be administered in an immediate release form to treat conditions or diseases associated with an increased IL-8 expression, such as chronic inflammatory conditions, including, without limitation, psoriasis, acute respiratory distress syndrome (ARDS), chronic obstructive pulmonary disease (COPD), rheumatoid arthritis (RA), as well as inflammations in various cancers in which IL-8 expression is increased. It may be concluded that ketamine and its metabolites serve as inflammation attenuators which decrease TNF-α and IL-6 secretion when provided for a prolonged time periods and thereby are effective in attenuating inflammation, caused by pathogenic infection, e.g., by fending off pathogenic infection, while limiting the degree of possible hyperinflammation.

In conclusion, all ketamines revealed anti-inflammatory effects with (R,S)-ketamine hydrochloride being the most potent ketamine However, long-term pre-stimulation improved the anti-inflammatory effects of (2R,6R)-hydroxynorketamine hydrochloride and (2S,6S)-hydroxynorketamine hydrochloride on TNF and IL-6, but all ketamines had no effect on IL-8. IL-1β was only weakly synthesized in 2- or 3-days old monocytes.

### Example 5—Treatment of SARS-CoV-2 Positive Patients with HNK

It has been found recently that in COVID-19 patients, IL-6 and TNF-α serum levels are direct, independent, and considered as inflammatory cytokine signature predictors of COVID-19 severity and survival (Del Valle DM, et al. (2020); Nature Medicine 26: 1636-1643). PGE2 is a pro-inflammatory cyclooxygenase-2 mediated metabolite of arachidonic acid. The concentrations of PGE2 are elevated in COVID-19 patients and increased PGE2 concentrations correlate with disease progression and decreased survival (Hong W, et al., (2020); Frontiers in Pharmacology 11: 561674; and U. N. Das (2021) Archives of Medical Research 52:1:107-120). Treatment of COVID-19 patients with the cyclooxygenase-2 inhibitor Celebrex significantly reduced PGE2 levels with positive therapeutic outcomes (Hong W, et al., (2020); Frontiers in Pharmacology 11: 561674). Thus, the ability of HNK to significantly reduce PGE2 production coupled with the attenuation of IL-6 and TNF-α production are predicted to produce positive effects in COVID-19 patients.

As to IL-8, this cytokine is a neutrophil chemotactic factor, which induces neutrophils to migrate toward the site of infection. IL-8 also stimulates phagocytosis once they have arrived. Neutrophils are a type of white blood cells (WBC or granulocyte) that protect the body from infections, among other functions. They make up approximately 40% to 60% of the white blood cells in the body and are the first cells to arrive on the scene of an infection. The serum levels of IL-8 are also significantly elevated in COVID-19 patients, but IL-8 serum levels are not an independent marker of disease severity or patient survival (Del Valle DM, et al. (2020); Nature Medicine 26: 1636-1643). As shown in the above graphs, long term treatment with HNK does not affect IL-8 production suggesting that HNK may not reduce the recruitment of neutrophils to SARS-CoV-2 infected tissues. This property coupled with reduced production of IL-6 and TNF-α indicates that HNK attenuates rather than completely eradicates or blocks the immune response to SARS-CoV-2 infection. This property of HNK may be of particular importance due to the proper balance required to maintain immune activity against the infection and at the same time attenuate the hyperinflammation response which may be harmful to the body.

SARS-CoV-2 positive patients (typically, but not necessarily mild to severe patients) are evaluated for the presence of elevated plasma and/or saliva concentrations of cytokines, such as interleukins (IL-1β), IL-6, IL-8, chemokines (e.g., PGE2) granulocyte colony-stimulating stimulating factors (G-CSF), and/or tumor necrosis factor-1α (TNF), and optionally also other markers of systemic inflammation.

The patient's temperature, blood oxygen levels, respiration rate and other determinants of clinical status are determined Patient anxiety and depression may be further assessed using the Hospital Anxiety and Depression Scale (HADS).

A pharmaceutical composition comprising HNK is administered to the patient (optionally a single dose treatment, a daily administration, continuous infusion, and/or in a prolonged treatment procedure) and the plasma or saliva concentrations of the inflammatory markers, general clinical status, and anxiety and depression are monitored. A positive response is defined as reduced plasma/saliva concentrations of inflammatory markers, improved clinical status and reduced anxiety and depression as measured by the HADS score. The HNK may be administered via IV infusion, IP (intraperitoneal) administration, by intranasal, sublingual, or by oral administration, at an optional dose, below the normal anesthetic dose (e.g., 0.15 mg/kg-0.3 mg/kg, based on total body weight for maximum 20 mg every 6 hours).

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

1-56. (canceled)

57. A method of inhibiting or attenuating proinflammatory agent secretion from immune cells, the method comprises contacting the immune cells with a composition comprising an effective amount of HNK, a stereoisomer thereof, a pharmaceutically acceptable salt thereof, or a combination thereof, thereby inhibiting or attenuating proinflammatory agent secretion from the immune cells.

58. The method according to claim 57, wherein the proinflammatory agent is selected from a group consisting of IL-6, TNF-a, IL-8, IL-Ib, prostaglandin E2 (PGE2) and a combination thereof.

59. The method according to claim 57, wherein the HNK is (2R,6R)-hydroxynorketamine, (2S,6S)-hydroxynorketamine, or a combination thereof.

60. The method according to claim 57, comprising contacting the immune cells with the HNK for at least about 30 minutes, at least about 12 hours, at least about 24 hours, or at least about 48 hours.

61. The method according to claim 57, wherein the concentration of the HNK is at least about 0.1 mM, at least about 10 μM, or at least about 50 μM.

62. The method according to claim 57, wherein the immune cells are monocytes and/or macrophages.

63. The method according to claim 57, wherein the immune cells are stimulated for enhanced proinflammatory agent secretion by a pathogen or a portion thereof, a trauma, a hazardous substance, or an autoimmune disease.

64. The method according to claim 57, wherein the pathogen is selected from the group consisting of a virus, a bacteria, a protozoa, a prion, a viroid, or a fungus.

65. The method according to claim 57, wherein the HNK attenuates proinflammatory agent secretion from stimulated immune cells by at least about 20% as compared to immune cells not subjected to treatment with HNK.

66. A method of treating or attenuating inflammation in a subject in need thereof, the method comprising administering to the subject a pharmaceutical composition comprising a therapeutically effective amount of hydroxynorketamine (HNK), a stereoisomer thereof, a pharmaceutically acceptable salt thereof, or a combination thereof, and a pharmaceutical acceptable carrier, thereby treating inflammation in the subject.

67. The method according to claim 66, wherein the HNK is (2R,6R)-hydroxynorketamine, (2S,6S)-hydroxynorketamine, or a combination thereof.

68. The method according to claim 66, wherein the pharmaceutical composition is for administration in a route selected from the group consisting of: oral, intravenous, intraperitoneal, intranasal, subcutaneous, sublingual, intrathecal, transdermal, buccal, vaginal, rectal, topical, ocular, otic, and a combination thereof.

69. The method according to claim 66, wherein the dosage of the HNK within the composition is within the range of from 0.01 mg to 5000 mg, from 1 mg to 5000 mg, from 1 mg to 1000 mg, from 1 mg to 500 mg, or from 10 mg to 200 mg.

70. The method according to claim 66, comprising a prolonged administration comprising a sustained release dosage form of the HNK, a prolonged administration time of the HNK, a repeated administration, or a combination thereof.

71. The method according to claim 70, wherein the prolonged administration time comprises HNK administration to the patient as an infusion over a period of 10 minutes to 48 hours, 10 minutes to 24 hours, 30 minutes to 12 hours, or 30 minutes to 4 hours.

72. The method according to claim 66, wherein said proinflammatory agent is selected from a group consisting of IL-6, TNF-a, IL-8, IL-Ib, prostaglandin E2 (PGE2), or a combination thereof.

73. The method according to claim 66, wherein the inflammation is caused by a pathogen, a trauma, a hazardous substance, or an autoimmune disease.

74. The method according to claim 73, wherein the pathogen is selected from the group consisting of a virus, a bacteria, a protozoa, a prion, a viroid, or a fungus.

75. The method according to claim 74, wherein the pathogen is a virus.

76. The method according to claim 75, wherein the virus is a SARS-CoV-2.
